(19) United States
(12) Patent Application Publication
(10) Pub. No.: US 2025/0079012 A1
(43) Pub. Date: Mar. 6, 2025

(54) IDENTIFYING MATERNITY SEVERITY INDEX FOR WOMEN

(71) Applicant: INNOVACCER INC., San Francicso, CA (US)

(72) Inventors: Ajay LAKSHMAN, Noida (IN); Mukul SONKER, Noida (IN); Vaibhav ., Noida (IN)

(21) Appl. No.: 18/540,583

(22) Filed: Dec. 14, 2023

(30) Foreign Application Priority Data
Aug. 31, 2023 (IN) 202311058557

(51) Int. Cl. G16H 50/30; G16H 15/00

(52) U.S. Cl. CPC G16H 50/30; G16H 15/00

(57) ABSTRACT

Determining a maternity severity index of a subject user includes providing at least one input query on a state of a user. An output module elicits a response from the user to the input query. A data receiving component receives datasets pertaining to risks associated with maternity from data sources. A data repository, including a risk assessment database having sample maternity data parameter, and data-sets are constantly upgraded with parameters, assessments, and recommendation plans generated for other patients. A risk assessment module determines a current risk persona of the subject user categorized in the form of risk-based clusters based on a response of the user to the query, the received data-sets, and the sample maternity data parameters. A series of predicted maternity risk realizations are calculated by correlating the response of the user with the sample maternity data parameters. A risk report is generated which determines a maternity risk possibility.

### CROSS REFERENCE TO RELATED APPLICATION

This application claims benefit of India application No. 202311058557 filed Aug. 31, 2023, the entire disclosure of which is incorporated herein by reference.

### TECHNICAL FIELD

The present subject matter generally relates to a maternity health improvement system and more particularly relates to a system for identifying risks related to maternity, and specifically maternity severity index.

### BACKGROUND

In today's world, there has been a vast increase in various problems related to maternity such as low fertility, abnormal child and unwanted abortions due to various complications before or during the pregnancy which were not timely attended to. Such an inevitable increase in high-risk pregnancies is majorly dependent on various health and/or socio-economic factors such as including but not limited to, late marriages and older pregnancies, ignorance of underlying behavioral or medical problems, and so on. High-risk pregnant women should be provided with a timely medical service compared to normal pregnant women due to direct connection with the death of newborns and maternal deaths. However, it is difficult to timely determine whether a subject patient is high risk patient or not since majority of such determination techniques considers only physical symptoms/tests such as heart disease, stroke, cancer, diabetes, and arthritis, without considering various behavioral/socio-economic factors. Accordingly, the risks are mitigated/migrated into unwanted events causing a large number of fetal as well as mother's death.

Generally, thorough prenatal care during pregnancy is critical as risk perception in relation to pregnancy and child birth is a complex process that is influenced by multiple personal, psychological and societal factors. Sometimes, negligence during pregnancy may lead to adverse maternal and fetal outcomes, including unnecessary cesarean sections, related post-surgical complication, inaccurate detection of fetal hypoxia and other fetal complications. Even normal pregnancies cause various discomforts and symptoms. It is often difficult to distinguish a normal pregnancy from a complicated one based on discomforts and symptoms. The major complications of preeclampsia, preterm birth, small for gestational age, intrauterine growth restriction and gestational diabetes mellitus occur in about 25% of first pregnancies. Therefore, the risk of maternal morbidity and mortality is of public concern worldwide. Many women die daily from preventable causes related to pregnancy and childbirth. However, thorough diagnosis and early detection/prediction of the complications during pregnancy period would be helpful in preventing the adverse effects. Early detection of life-threatening complications could be supportive in initiating targeted intervention at an early stage and improve the benefits of medical/nutrition therapy. Patients might also respond better to various types of lifestyle and pharmacological preventative interventions.

However, in order to enable targeted interventions towards those at high-risk, whilst avoiding exposure of those at low risk to the potential intervention-associated, many studies have deployed the risk factors associated with prenatal risks, which are used to calculate the risk score index (RSI). The RSI reflects the combined risk of existing maternal variables to identify women at different levels of risk for adverse pregnancy in order to prevent maternal and fetal complications. Generally, the RSI identifies low, moderate, and high-risk pregnancies, where the higher scores were associated with maternal complications as well as neonatal mortality and morbidity. Further, various factors are considered while calculating RSI such as maternal demographics, mortality, stillbirths, male gender, and congenital anomalies and their associations with categories of pregnancy level of risk. But the factors considered should be updated and validated for an accurate score. Sometimes the response to the factors is not updated on regular basis or neglected then it may result in erroneous output which may further lead to high risk pregnancy. Therefore, systems have been designed to avoid any manual negligence of the information of the patient. Further, managing, validating and updating patient history data becomes cumbersome for huge number of patients.

Accordingly, it is desirable to provide a system that could determine a patient's maternity severity index in advance and also predict various risks associated with the pregnancy, so that such risks may be timely taken care of.

### SUMMARY

In one aspect of the present subject matter, a system for determining maternity severity index of a subject user is disclosed. The system includes a computing unit communicably connected to a backend server comprising an input component, adapted to present and/or formulate at least one input query based at least in part on one or more health and/or socio-economic and/or behavioral states of said subject. The computing unit further includes an output module adapted to elicit at least one response from said subject to the presented input query. The backend server includes a data receiving component adapted to receive one or more datasets pertaining to at least one risk associated with maternity, from one or more data sources. The system further includes a data repository connected to the computer and consisting of a risk assessment database, comprising a plurality of data sets such as for example, related to various kind of sample maternity data and/or parameters and/or features, assessments/evaluations of various risks related to maternity, suggested recommendations and/or avoidance plan for treating/avoiding said risks from taking place, from a plurality of users of different age, demographic regions, races, origin and the like. The data repository includes the risk assessment database and the plurality of data-sets are constantly upgraded with parameters, assessments, and recommendation plans generated by the system for other patients. Moreover, the system includes a risk assessment module comprising a set of programming instructions generally based on one or more deep learning model, and configured to determine a current risk persona of the subject user categorized in the form of one or more risk-based clusters, on the basis of response of the subject to the query, the received data-sets, and the sample maternity data parameters, calculate a series of predicted maternity risk realizations for a predetermined future time span by at least partially correlating the response of the subject to the query with the sample maternity data parameters, generate a risk report for the predetermined time period, report at least determining a risk possibility of a preterm birth and/or a cesarean delivery at the point of the birth time.

Preferably, the one or more query includes questions related to but not limited to mental/behavioral states, physical/medical states, and socio-economic condition of the subject user.

Additionally, the one or more queries include a plurality of queries presented in a sequential manner to the subject and configured to systematically elicit said plurality of responses from the subject.

In another aspect of the present disclosure, the present disclosure provides a method for using the maternity severity index identification system for the purpose of risk-based clustering of a subject patient. The method includes the step presenting one or more queries to the subject patient on an interface on a computing unit. The method further includes the step of receiving response to the one or more queries from the subject patient from the computing unit to a backend server. Furthermore, the method includes the step of receiving one or more data-sets pertaining to at least one risk associated to maternity within the subject patient.

The method furthermore includes the step of receiving one or more data-sets pertaining to at least one risk associated to maternity within the subject patient. Additionally, the method includes the step of utilizing a maternity risk assessment module for the purpose of determining a current risk persona of the subject patient on the basis of response of the subject to the query, the received data-sets, and a risk assessment database comprising a plurality of sample maternity data parameters and generating a risk report for the predetermined time period, the report at least determining a risk possibility of a preterm birth and/or a cesarean delivery at the point of the birth time.

In yet another aspect, the present disclosure provides a method of using the risk assessment module for the purpose of calculating a series of predicted maternity risk realizations for a predetermined future time span by at least partially correlating the response of the subject to the query with the sample maternity data parameters.

In yet another aspect, the present disclosure provides a method of generating an intervention strategy to avoid realizations of predicted risks related to maternity within the subject patient. The method includes the step of generating a maternity conditional recovery plan including one or more recommendations/health plans/medications/lifestyle changes targeted towards reducing the possibility of risks and avoidance of the migration of risks between different trimesters of maternity period.

The details of one or more implementations are set forth in the accompanying drawings and the description below. Other aspects, features and advantages of the subject matter disclosed herein will be apparent from the description, the drawings, and the claims.

### BRIEF DESCRIPTION OF THE DRAWINGS

FIG. 1 is a high-level block diagram of a maternity severity index identification system in accordance with embodiments of the present subject matter.

FIG. 2a is a high-level block diagram of a maternity severity index identification system in accordance with embodiments of the present subject matter.

FIG. 2b is a block diagram displaying maternity risk assessment sub modules in accordance with embodiments of the present subject matter.

FIG. 3 is a data flow diagram illustrating a data flow from input query request to a subject patient maternity risk index identification in accordance with embodiments of the present subject matter.

FIG. 4 is a flowchart illustrating the steps for treating the subject patient, in accordance with another exemplary clinical use case of the present subject matter.

FIGS. 5a & 5b illustrates an exemplary determination of maternity severity index for a set of subject patients, in accordance with embodiments of the present subject matter.

FIG. 6 illustrates an exemplary intervention strategy determination for monitoring the pregnancy of a set of subject patients in accordance with embodiments of the present subject matter.

FIG. 7 illustrates an exemplary process of segmenting eligible employees using pre-existing clinical factors in accordance with embodiments of the present subject matter.

### DETAILED DESCRIPTION

As required, a schematic, exemplary-only embodiment of the present application is disclosed herein; however, it is to be understood that the disclosed embodiment is merely exemplary of the present disclosure, which may be embodied in various and/or alternative forms. Specific structural and functional details disclosed herein are not to be interpreted as limiting, but merely as a basis for the claims and as a representative basis for teaching one skilled in the art to variously employ the present disclosure in virtually any appropriately detailed structure.

Aspects, advantages and/or other features of the exemplary embodiment of the disclosure will become apparent in view of the following detailed description, which discloses various non-limiting embodiments of the disclosure. In describing exemplary embodiments, specific terminology is employed for the sake of clarity. However, the embodiments are not intended to be limited to this specific terminology. It is to be understood that each specific portion includes all technical equivalents that operate in a similar manner to accomplish a similar purpose.

Exemplary embodiments may be adapted for many different purposes and are not intended to be limited to the specific exemplary purposes set forth herein. Those skilled in the art would be able to adapt the exemplary-only embodiment of the present disclosure, depending for example, on the intended use of adapted embodiment. Moreover, examples and limitations related therewith brought herein below are intended to be illustrative and not exclusive. Other limitations of the related art will become apparent to those of skill in the art upon a reading of the following specification and a study of the related figures.

The present disclosure relates to a maternity severity index identification system for determining presence of various risks related to maternity within a subject patient and thereafter, visualizing such risks mitigated across the maternity cycle of the subject patient for a period of time on a time-line scale. The system is further adapted to predict risk of Preterm Birth, and Cesarian at the time of birth. Furthermore, the system is adapted to recommend various measures such as health plans, medicinal plans, lifestyle changes, exercises, food habits, and the like to reduce/treat/avoid occurrence of such risks within the subject patient. The system generally is provided with a computing unit adapted to receive response to queries and/or surveys from the subject patient. In an embodiment of the present disclosure, the apparatus may be used as a web-based automated service. In another embodiment of the present disclosure, the apparatus may complement to clinics/doctors in determining such maternity issues along with a recommendation plan, which the doctors may customize as per their experience and requirements, so as to treat the underlying problems causing such maternity risks in the subject patient. It is to be understood that unless otherwise indicated, this disclosure need not be limited to applications related to determination of risks for a patient currently at any stage of maternity. As one of ordinary skill in the art would appreciate, variations of the disclosure may be applied to any kind of patient for any other possible disease, such as treatment of pain syndromes, mood disorders, various kind of rehabilitation programs, and the like. Moreover, it should be understood that embodiments of the present disclosure may be applied in combination with various other management systems such as employee management system, patient management systems, and the like, for various other possible applications. It must also be noted that, as used in this specification and the appended claims, the singular forms “a,” “an” and “the” include plural referents unless the context clearly dictates otherwise. Thus, for example, the term “a query” is intended to mean a single query or a combination of queries, “an algorithm” is intended to mean one or more algorithms for a same purpose, or a combination of algorithms for performing different program executions.

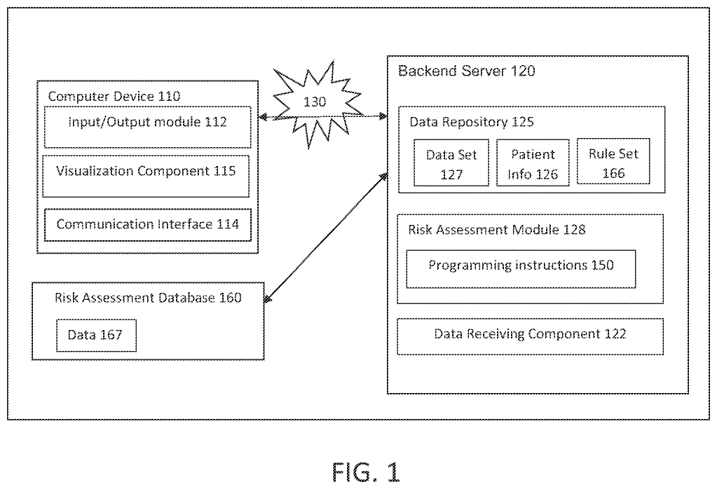

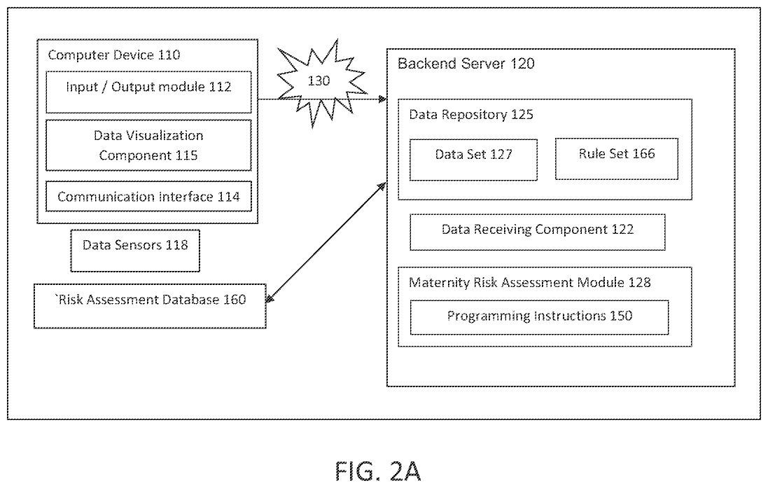

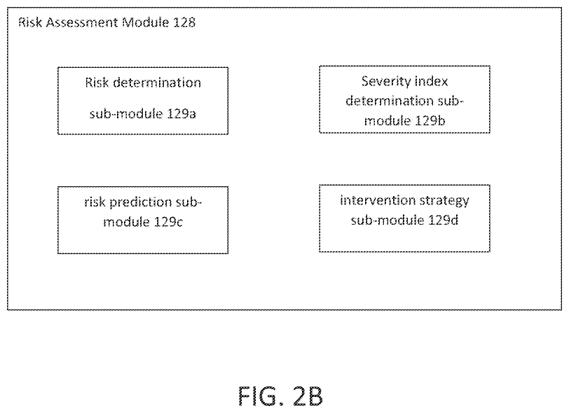

In description of the FIGS. 1-2 that follow, elements common to the schematic system will have the same number designation unless otherwise noted. In a first embodiment, as illustrated in FIG. 1, the present subject matter provides a system environment for a maternity severity index identification system 100 in accordance with some embodiments. As illustrated in FIG. 1, the system 100 includes a computing device 110 connected to a backend server 120 through a communication medium 130. While the system 100 illustrates only one computer device 110, it is contemplated for a person skilled in the art that the system environment can have any number of computer devices 110 as well as multiple systems 100 connected to each other.

The computing unit 110 includes an input/output module 112 adapted to present/formulate at least one query based at least in part on one or more of health related states and/or behavioral states and/or socio-economic state of a subject patient [not illustrated]. The input/output module 112 is further configured to elicit a response to the at least one query from the subject patient. The computing unit 110 can be personal or mobile computing devices, such as smartphones, tablets, or notebook computers. In some embodiments, the computing unit 110 may include an executable client application that uses an application programming interface (API) to communicate with the backend server 120 through the communication medium 130. The one or more queries may include questions related to subject patient's medical condition, various subjective symptoms (palpitations, swelling, and fatigue) Etc.), diet (type, intake, regularity, etc.), exercise (type, intensity, frequency, etc.), smoking (presence/absence, amount of smoking, years of smoking, past smoking history, smoking cessation period, etc.), alcohol consumption (frequency), Type, amount of alcohol, etc.) and daily life habits such as work (work contents, working hours, return time, etc.). in addition to any question that the healthcare professional deem as an important factor in determining the maternity risks that may be inherited, or the subjective, or the underlying mental/physical problems that may be faced by the subject patient.

The computing unit 110 further includes one or more communication interfaces 114 adapted to enable communication thereof with other stake holders, such as doctors, family, and the like, to whom an alert may be sent and/or to communicatively connect the computing device 110 to the back-end server 120 through the communication medium 130. In a preferred embodiment, the communication interface 114 is a high energy communication interface, generally in the form of a Wi-Fi interface, adapted to communicate with the back-end server 120 through the communication medium 130, generally in the form a network selected from one or more of but not limited to a WAN, Internet, Intranet, and the like. However, in other embodiments, the communication interface 114 may be in the form of a wired interface such as LAN, and the like.

In some embodiments, the system 100 may further include one or more data sensors 118 adapted to be placed in contact with the subject patient and configured to capture at least one physical/biological parameter related to the subject patient. In a preferred embodiment, the one or more data sensors may be any conventionally known data sensor suitable to sense at least one data type selected from the group comprising psychological data, reaction data, physical activity data, biosensor data, cognitive data, and emotional data. The data from the data-sensors 118 may be clubbed with the query response form and sent to the back-end server 120 via the computing unit 110.

In some embodiments, as illustrated in FIG. 2a, the one or more medical data sensors 118 may be any conventionally known medical data sensor suitable to sense at least one data type selected from the group consisting of user's temperature data, user's physical activity data, heart rate, HR variability, respiratory rate, biosensor data, cognitive data, pulse rate, galvanic skin response, and other specific indicators of Chronic/cardio logical symptoms (e.g. cough, changes in the user's voice, breathe sensors, behavior patter, and the like). It is to be understood that any different numbers, combinations and arrangements of sensors 118 could be used without deviating from the scope of the disclosure.

The backend server 120 is generally a computing unit having one or more data-receiving component 122 adapted to receive real-time data-sets 115 from a plurality of computing units 110. The data-sets 115 pertains at least to a response provided by the subject patient to the query form and/or real-time data-sets sensed from one or more of the plurality of data sensors 118.

The backend server 120 further includes a data repository 125 connected to one or more patient information sources 126 and comprising a plurality of data-sets 127 pertaining to medical and/or other related information for various chronic diseases. The plurality of data-sets 127 generally includes the medical history of the patient as well as current records of the monitored parameters, which in turn plays a central role in the determining the risks associated with the subject patient as well as to correlate the data received from the computing unit 110. In a preferred embodiment, the plurality of data sets 127 includes de-identified medical claims data for women in the age group of 18-40 for the year of pregnancy, a year prior to pregnancy and a year after pregnancy to cover the entire spectrum of pre-pregnancy, during pregnancy and post pregnancy. The de-identified claims data sets 127, in such embodiments, is used to predict high-risk maternity events, specifically C-section deliveries and non-routine neonatal intensive care unit (NICU) care. Further, in some embodiments, the data sets 127 includes social determinants of health to indicate regional social vulnerability risk in areas such as access to care, education, access to transportation, socio-economic index, racial disparities etc. to identify non-clinical factors that could impact a woman's health. For example, according to an embodiment of the present disclosure, when the subject patient visits the doctor, the diagnosis and symptoms as identified and/or monitored by the doctor during the visit, is fed to the computing unit 110 in real time to calculate/predict the risk. The fed data set 127 is received by the backend server 120. Such data set 127 is constantly updated on every visit of the subject patient. The backend server 120 further includes a maternity risk assessment module 128 to determine various risks associated with the maternity in the subject patient, and to cluster the risk persona in one or more indexing criteria.

The backend server 120 further includes a risk assessment database 160 comprising a plurality of sample maternity data parameters set 167 such as for example, including but not limited to a plurality of maternity related risks, disease and/or features dataset, health assessment reports comprising assessments of disease/symptoms within the user, remedial and/or treatment dataset comprising suggested intervention plans and/or recommendations and/or lifestyle plan for avoiding occurrence of such risks within the users. The sample maternity data parameters data set 167 comprises historical database from a plurality of users of different age, demographic regions, races, origin, socio-economic, biological considerations, and various other similar variations. In some embodiments, the remedial data-sets may be collected from the external sources, such as health care providers and research institutes, and/or the clinical data accumulated by the public health recovery institutes. In some embodiments, the maternal and paternal pre-conception data may be collected even before planning for the pregnancy, which may be helpful in identifying future complications that may arise due to the current lifestyle of the subject patients. The pre conception data may include the presence of chronic bronchitis, severe intestinal disorder, epilepsy, heart conditions, high blood pressure, diabetes, HIV/aids etc. Similarly, paternal characteristics such as paternal age, ethnicity, educational level, smoking and alcohol consumption may be collected, height, weight and systolic and diastolic blood pressure. In some embodiments, the risk assessment database 160 is positioned onto the backend server 120. However, in some other embodiments, the risk assessment database 160 is a cloud-based database connected to the plurality of backend servers 120 of the current system environments as well as other data sources [not shown].

The maternity risk assessment module 128 is configured to process the received data sets 115 in accordance with one or more programming instructions 150 and in accordance with a disease rule engine defined by a threat rule-set 166, defining one or more rules for estimating the maternity related risks within the subject patients, using the received data-sets 115, and the risk assessment database 160. The data repository 125 includes the risk assessment database 160 and the plurality of data-sets 167 are constantly upgraded on the basis of one or more learning models selected from but not limited to Natural language processing (NLP), Deep Learning, Machine Learning, statistical learning model, and the like.

In an embodiment, as illustrated in FIG. 2, the risk assessment module 128 includes a plurality of sub modules 129, each associated to a predetermined perspective of the risk assessment module 128. For example, in some embodiments, the sub modules 129 includes a risk determination sub-module 129a, a severity index determination sub-module 129b, a risk prediction sub-module 129c, and an intervention strategy sub-module 129d. Each of the sub-module 129 is associated to the predetermined programming instructions 150 embodied onto the memory and adapted to perform an action specific to the predetermined perspective thereof.

The set of programming instructions 150 are generally based on one or more deep learning model and configured to cause the backend server 120 and/or the computer 110 to determine a current risk persona of the subject user categorized in the form of one or more risk-based clusters, on the basis of response of the subject to the query, the received data-sets, and the sample maternity data parameters. The programming instructions 150 are further configured to calculate a series of predicted maternity risk realizations for a predetermined future time span; by at least partially correlating the response of the subject to the query with the sample maternity data parameters. The programming instructions 150 are furthermore configured to generate a risk report for the predetermined time period, the report at least determining a risk possibility of a preterm birth and/or a cesarean delivery at the point of the birth time by at least partially correlating the response of the subject to the query with the sample maternity data parameters of the risk assessment database.

In preferred embodiments, the deep learning model may be based on one or more of below mentioned models:—

Gaussian Mixture Models (GMM): This model is used for clustering by specifying the number of clusters and randomly assigning data points to each cluster. Then, the parameters of the GMM are estimated using Expectation-Maximization (EM) Algorithm. The EM algorithm iteratively updates the cluster assignments and recalculates the parameters, such as the means, covariances, and mixing coefficients, to maximize the likelihood of the data given in the model. Further, in order to evaluate quality of clustering, silhouette score for each data point is calculated. The silhouette score quantifies similarity of a data point to its assigned cluster compared to other clusters. The score ranges from −1 to 1, where values closer to 1 indicate better cluster assignments.

K-means Algorithm: The methodology is used by specifying the number of clusters and randomly initializing cluster centroids. The K-means algorithm iteratively optimizes the cluster assignments and updates the centroid positions. In each iteration, it assigns each data point to the nearest centroid and recalculates the centroids based on the assigned data points.

In order to evaluate the clustering performance, a Calinski-Harabasz (CH) Score is calculated. The CH score compares the between-cluster variance to the within-cluster variance, providing an indication of cluster compactness and separation. The higher CH scores indicate better-defined and well-separated clusters.

In an embodiment of the present disclosure, the system 100 including the programming instructions 150 and the implementation to process the data-sets 115 and/or the data repository 125 are based on a deep learning model wherein the model is particularly applied to upgrade the data repository 125 including each of the data sets 127 and 167. Particularly, the deep learning model in such embodiments may include a number of pre-processing steps that are applied on the data stored in the all the individual data sets 115, 127, 167. The pre-processing steps may include cleansing the data to remove any inconsistencies and assigning weights to each of the parameters for the consideration of assessments. Further, in such embodiments, the data sets 115, 127, 167 may be preprocessed by handling missing values, encoding categorical variables, and performing feature scaling or normalization. Particularly, a list of parameters/features may additionally be determined at this step. In some embodiments, the step of preprocessing may include identification of relevant features, such as a combination of International Classification of Diseases (ICD) codes and Z codes. The ICD codes identify the occurrence of C-section deliveries and non-routine NICU care, providing a binary classification of event presence, thereby allowing determination of whether an event occurred or not. Additionally, Z codes are utilized to establish the chronology of events within a year. By leveraging Z codes, which include codes representing the onset of pregnancy and other pregnancy-related events, a comprehensive timeline of events is constructed. This approach overcomes the limited temporal information available in claims data and enables a detailed understanding of the sequence of events.

Further, the machine learning model and/or the deep learning model includes a learning engine adapted to run a selected model (e.g., GMM or K algorithm model, deep learning model, Random Forest, multi linear regression, Multilayered, feed-forward neural networks, statistical model or the like) on the data sets 115, 127, 167, and partitions them into either a training dataset or a testing dataset. In a preferred embodiment, the partitioning may apply an 80/20 split between the training dataset and the testing dataset, respectively.

Thereafter, the learning engine operates to then run the selected model on the training dataset to obtain a resulting output from the model. For example, in a preferred embodiment, the selected model is the Multilayered, feed-forward neural networks, with a Tensor flow backend to build and train the neural networks.

The learning engine then selects and tunes other model arguments of the training dataset to establish an error percentage. Once the error percentage (i.e., accuracy) is established, the learning engine applies a ten-fold cross validation to establish a model stability of the selected model. Further, the learning engine operates dynamically by dynamically selecting the model arguments for each run of the selected model.

Further, the learning engine operates a final model run on the testing dataset to confirm the accuracy and/or fit of the selected model are within client acceptable limits. When the accuracy and/or fit of the selected model is not within the client's acceptable limits or when there are more models left for consideration, a next model may be selected to begin the testing process over again. When the accuracy and/or fit of the selected model is determined to be within the client acceptable limits or when there are no more models left for consideration, selected model is established for use to predict risk in maternity and/or intervention strategy for one or more subject users of the computing unit 110.

In certain other embodiments, the programming instructions 150 may be based on any predetermined threat assessment model selected from a statistical model (e.g., linear regression, non-linear regression, Monte Carlo simulation), heuristic models (e.g., neural networks, fuzzy logic models, expert system models, state vector machine models useful in risk and safety prediction), and so on, that may be used to predict a possible maternity related health risk, well in advance.

For example in an embodiment, the data sets 127 and 167 include de-identified medical claims data for women in the age group of 18-40 for the year of pregnancy, a year prior to pregnancy and a year after pregnancy to cover the entire spectrum of pre-pregnancy, during pregnancy and post pregnancy and social determinants of health to indicate regional social vulnerability risk in areas such as access to care, education, access to transportation, socio-economic index, racial disparities etc. to identify non-clinical factors that could impact woman's health. In such an embodiment, the preprocessing steps include using a programming language, such as Python, to transform the data by removing null values and removing multiple pregnancy episodes within the same year. Alternatively, the dataset 127 and 167 are preprocessed by handling missing values, encoding categorical variables, and performing feature scaling or normalization. For every pregnant woman, the date of pregnancy announced/detected via the claim is the index date for pregnancy and the delivery date is the date of delivery. Further, the method includes creating features to understand risk profile prior to pregnancy, by pulling a predetermined time period such as for example, 12 months, prior to pregnancy date's data to create features such as comorbid conditions, healthcare utilization (inpatient utilization, O/P utilization, medications), demographics (deidentified, such as age group) and leveraged social determinants at a region level by using 3 digit zip as a pseudo regional identifier and collate all zip level social determinant data to identify possible social risks. Thereafter, identification of pregnancy related complications or adverse events, is done to create features, such as advent of miscarriage, non-urgent NICU, non-urgent CSEC, which is the universe of adverse events in an embodiment. Furthermore, in such embodiments, the identification of upcoming risks during pregnancy, such as diseases like, gestational diabetes, preeclampsia etc. may be identified, which in turn may have an impact on the pregnancy.

According to an exemplary embodiment, distinct set of parameters (profiles) for clustering of patients are created. The first set of parameters focuses on historical clinical conditions, social factors, and prior pregnancy-related complications and indicates how the factors influenced the risk of adverse outcomes. Claims utilization for pre-existing diagnostic conditions, such as mental health disorders, infertility, obesity, chronic conditions, family conditions like cystic fibrosis and sickle cell anemia, is analyzed. This analysis allowed for the identification of patients at higher risk of adverse outcomes and provides insights into the potential effects of these conditions on maternal health and pregnancy outcomes.

The second set of parameters is based on clinical conditions and medical utilization patterns. Factors such as preventive care visits, medication usage, and office visit patterns are examined to explore the correlation between clinical factors and the eventual outcome. Procedures performed during the antenatal period, such as non-invasive prenatal testing (NIPT) and quad screenings, are also considered to provide additional information on potential risks and complications. The second set of parameters indicates the influence of clinical conditions and medical utilization patterns on high-risk maternity events.

In addition to the pre-pregnancy clustering, the profiles based on complications during pregnancy are created based on healthcare facility utilization and received treatment.

The identification of features involves the use of ICD and Z codes to determine the occurrence and chronology of C-section deliveries and non-routine NICU care. Patient profiles are created based on historical conditions, social factors, prior pregnancy-related complications, and clinical characteristics. By analyzing the profiles and comparing the profiles during different phases, valuable insights are gained into the factors influencing high-risk maternity events.

That is, the learning model is trained to predict various kind of risks that may be present/occur during different stages of the maternity by analyzing the submitted response to queries as well as data collected by the one or more sensors, and on the basis of sample historical data from a plurality of other subject patients and/or users.

The system 100 additionally includes a visualization component 170 configured as a GUI adapted to visualize the maternity risk clustered in form of severity index along with a series of expected trimester wise risk during various stages of maternity, on a time-line scale. In a preferred embodiment, the time-line scale is a graphical interface designed in the form of monthly segments, each monthly segment adapted to visually risks predicted to occur within the corresponding month. In another embodiment, the time-line scale is a graphical interface designed in the form of various stages of maternity such as including, a prematernity, first trimester, a second trimester, third trimester, a post pregnancy period, each stage displaying the risk predicted to occur within the corresponding trimester. Separate predictive models are tailored to forecast complications, such as placenta previa, preeclampsia, gestational diabetes, preterm birth, syphilis, and maternal depression.

The maternity risk assessment module 128 is further adapted to utilize the programming instruction set 150 to generate an intervention strategy adapted to reduce the possibility of occurrence of risky events as predicted for the subject patient. Particularly, the intervention strategy includes a plurality of recommendations and/or lifestyle/medical/health plans suitable for the subject based on the predicted risks for the subject patient. Further, the maternity risk assessment module 128 continuously evaluates the effectiveness of interventions, thus empowering healthcare providers to make informed decisions. If the risk of adverse events remains unchanged or increased over time despite treatment as per the intervention strategy, a scoring mechanism is facilitated for the identification of alternative treatment plans. The approach not only rectifies issues of ineffective undertreatment but also contributes to a reduction in overtreatment by identifying cases where the risk of adverse events significantly diminishes, following the treatment.

The functions performed by the various entities of FIG. 1 may vary in different embodiments. For example, in some embodiments, the computing unit 110 is operated by the subject patient itself, to self-realize the risk that may be faced during the maternity period. However, in other embodiments, the computing unit 110 may be used by a health professional to receive data from the subject patient, and the system, 100, may be used for suggestions/testing purpose only.

Although illustrated and described herein as computing unit 110, it is to be understood that embodiments of the disclosure could be implemented with any kind of computing devices, such as a laptop computer, smartphone, or the like. In another embodiment, a healthcare professional administering the tests enters the subject's answers via the computing unit 110. Similarly in some other embodiments, the computer unit 110 may be embodied with additional input/output components which may be contemplated by a person skilled in the art without deviating from the scope of current disclosure.

Similarly, the data sensors 118 may be embodied in a plurality of ways. For example, in some embodiments, the data sensors 118 may be in form of wearable devices having one or more sensors for detecting one or more of the subject's pulse rate, temperature, galvanic skin response, temperature, movement, etc. However, in other embodiments, sensors 118 may be in form of non-wearable devices comprising one or more imaging sensors of a camera, for example, for collecting image data representative of the subject's facial expression, skin temperature, and/or the like. In yet another embodiment, the sensors 118 comprise one or more electroencephalography (EEG) sensors in a headset, for example, for detecting the subject's EEG response. Moreover, in additional embodiments, the data sensors 118 may include sensing psychological reactions, measure the subject's non-verbal psycho-physical/psychological-physical stress and behavior reaction when responding to the one or more queries, or otherwise in general when seating in front of the healthcare professional. It is to be understood that any different numbers, combinations, and arrangements of sensors 118 could be used without deviating from the scope of the disclosure.

FIGS. 1 and 2 schematically show the arrangement of the basic components of the system of the present subject matter. However, in the construction of automated tools, secondary components such as different memories, databases, clouds, network and other related functional components known to one of skilled in the field of automated tools based on deep leaning methodologies, may be incorporated in the system. Such functional and/or supporting components are included in the present disclosure as long as the logical components and arrangements disclosed herein are present.

In an embodiment of the present disclosure, the backend 120 is programmed to implement different methods of the disclosure. The backend server 120 is programmed or otherwise configured to assess the subject patient's response to one or more queries related to the maternity risk of the subject patient in a single session or over multiple different sessions through the computing unit 110. The computing unit 110 in such embodiments is configured to regulate various aspects of assessing the different state of the subject in a single session or over multiple different sessions of the present disclosure, such as, for example, presenting queries, retrieving data, and processing data, and forwarding to the backend server 120. Further, the back-end server is adapted to receive a feedback response after a predetermined time period of intervention strategy and provide an updated risk clustered report for the subject patient.

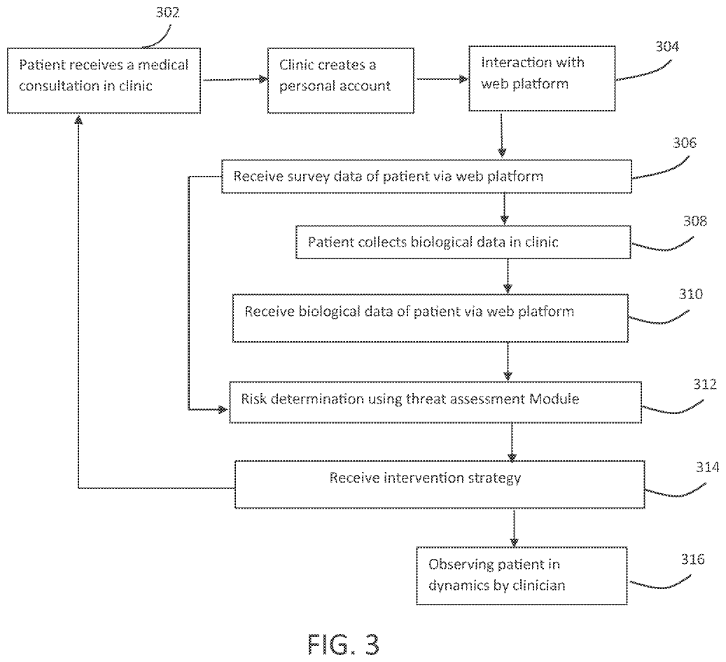

FIG. 3 illustrates a data flow diagram illustrating a data flow from input query request to a subject patient psychological age prediction. In a first embodiment, the subject patient receives a medical consultation in a clinic of healthcare specialist. At a step 302, the method includes creating a user's account onto the application programming interface (API) so as to enable a connection to the central server therethrough at step 304. The method proceeds to step 306 where the subject patient is presented with the one or more queries to which it responds through the API. The method then proceeds to an optional step 308 where the system detects one or more sensor data from the subject patient. Alternatively, the healthcare professional may collect the one or more biological data manually within the clinic. In another embodiment of the present disclosure, the clinics/doctors may determine maternity issues along with a recommendation plan, which the doctors may customize as per their experience and requirements, so as to treat the underlying problems causing such maternity risks in the subject patient. The sensed, recommended and/or collected data is then transferred to the backend server through the API at the step 310. At step 312, the set of programming instructions are executed by the risk assessment module 128 of the backend server 120 to determine various maternity related risks within the subject patient. In some embodiments, the risks are clustered in form of severity index either in a numeric or a word based category. Thereafter, at step 314, the system provides an intervention plan on the basis of deep learning model based optimized rules set, which the healthcare professional may share with the user either as is or otherwise along with his own customizations and/or feedbacks. For example, according to an embodiment of the present disclosure, when the subject patient visits the doctor, the diagnosis and symptoms as identified and/or monitored by the doctor during the visit, is fed to the computing unit 110 in real time to calculate/predict the risk. The fed data set 127 is received by the backend server 120. Such data set 127 is constantly updated on every visit of the subject patient. The method then proceeds and moves back to step 306 where the subject patient provides a follow back feedback after following a predetermined time period and follows the whole method to determined updated maternity risk index along with updated intervention strategy. Such a follow back is followed till the maternity of the subject patient is completed.

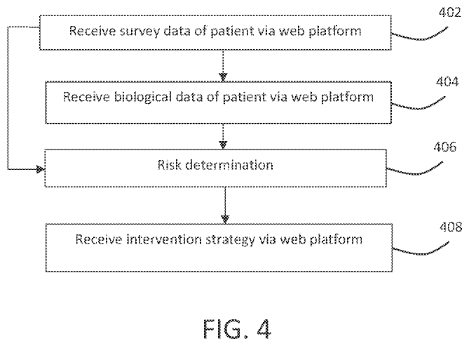

In an alternative embodiment as illustrated in flow diagram of FIG. 4, the subject patient first uses the system 100 to determine various maternity risks, along with severity index and the intervention strategy to avoid such risks from happening and/or migrating into different stages of the maternity. Thereafter, the subject patient consults with the healthcare professional to receive his feedback/consultation on the basis of the same.

Accordingly, the system 100 may be used for determining maternity severity index as well as future realization of risks related to maternity for the subject patient to determine various concerns associated thereto. Further, the system 100 may be used to determine an intervention strategy using deep learning methodologies to treat the determined problems, either medicinal/behavioral and/or in lifestyle of the subject patient.

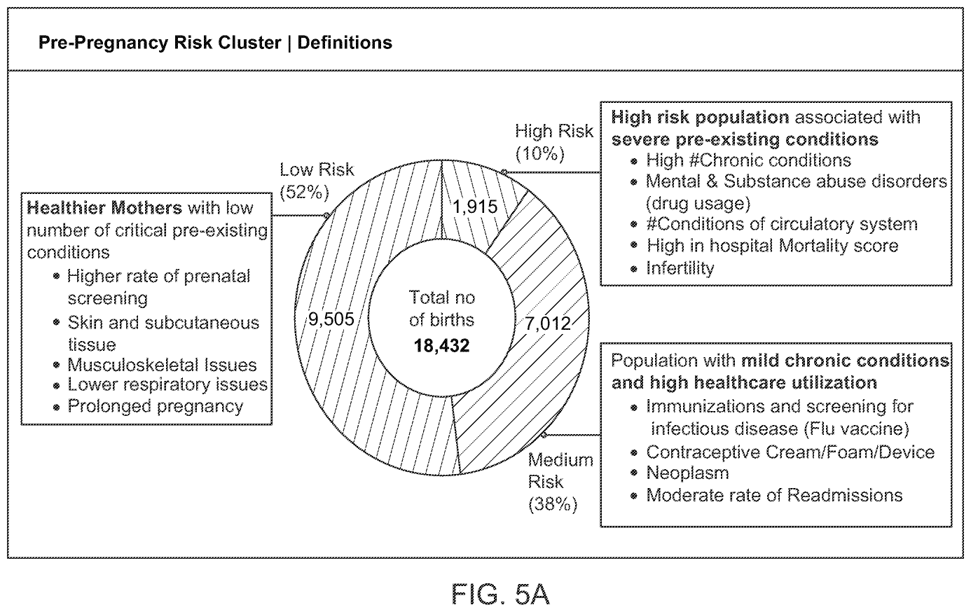

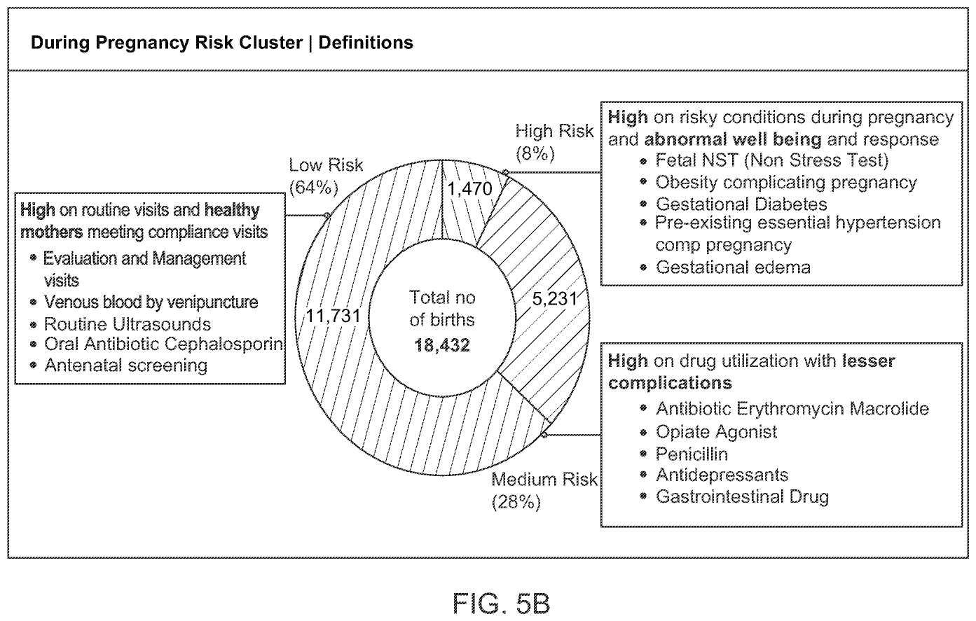

FIGS. 5a and 5b illustrate an exemplary determination of maternity severity index for a set of subject patients, through different phases of pregnancy according to an embodiment herein. According to an embodiment, the maternal and paternal pre-conception data may be collected even before planning for the pregnancy, which may be helpful in identifying future complications that may arise due to the current lifestyle of the subject patients. The pre conception data may include the presence of chronic bronchitis, severe intestinal disorder, epilepsy, heart conditions, high blood pressure, diabetes, HIV/aids etc. Similarly, paternal characteristics such as paternal age, ethnicity, educational level, smoking and alcohol consumption may be collected, height, weight and systolic and diastolic blood pressure. According to another embodiment of the disclosure, the data of the user may be collected at a very early stage of life which may be used to predict the future risks of the user. As illustrated, the risks are determined and clustered in the form of a pre-pregnancy risk cluster (as shown in FIG. 5a) and at the time of pregnancy risk cluster (illustrated in FIG. 5b). The risk cluster comprises a high risk, medium risk and low risk. In pre-pregnancy risk cluster, the high-risk population comprises severe pre-existing conditions like high chronic conditions, mental and substance abuse disorders, condition of circulator system, high in hospital mortality score and infertility. The medium risk population comprises mild chronic conditions and high healthcare utilization like immunization and screening for infectious disease, contraceptive cream/foam/device, neoplasm, moderate rate of readmissions. The low-risk population comprises healthier mothers with low number of critical pre-existing conditions like high rate of prenatal screening, skin and subcutaneous tissue, musculoskeletal issues, lower respiratory issues and prolonged pregnancy. The preconception period represents an opportunity to intervene and improve outcomes. If the intervention with proper medication and improved lifestyle is provided in the pre pregnancy stage, the high risk factors may be controlled to a normal low risk pregnancy. Subject patient's preconception health includes modifiable risk factors that stands to reduce the incidence of adverse pregnancy complications and birth outcomes. As illustrated in FIG. 5a, during pre-pregnancy, the risk cluster represents 52% healthier mothers with low number of critical pre-existing conditions, which clearly signifies that intervention during pre-pregnancy period resulted in the low risk to the subject patient and child birth during later stage of pregnancy. Further, 38% mothers who were provided proper intervention due to predicted risks during pre-pregnancy period, had mild chronic conditions and high healthcare utilization. Only 10% who were associated with severe preexisting conditions were left with high risk. However, such conditions are also handled with better interventions if identified during earlier years of life. The high-risk condition during pregnancy, abnormal well-being and response comprises fetal NST, obesity complicating pregnancy, gestational diabetes, pre-existing essential hypertension comp pregnancy and gestational edema. The medium risk during pregnancy occurs on drug utilization with lesser complications that includes antibiotic erythromycin macrolide, opiate agonist, penicillin, antidepressants and gastrointestinal drug. The low risk during pregnancy occurs on routine visits and healthy mothers meeting compliance visits includes evaluation and management visits, venous blood by venipuncture, routine ultrasounds, oral antibiotic cephalosporin and antenatal screening. In some embodiments, during pregnancy the high-risk factor is 8%, medium risk factor is 28% and low risk factor is 64%.

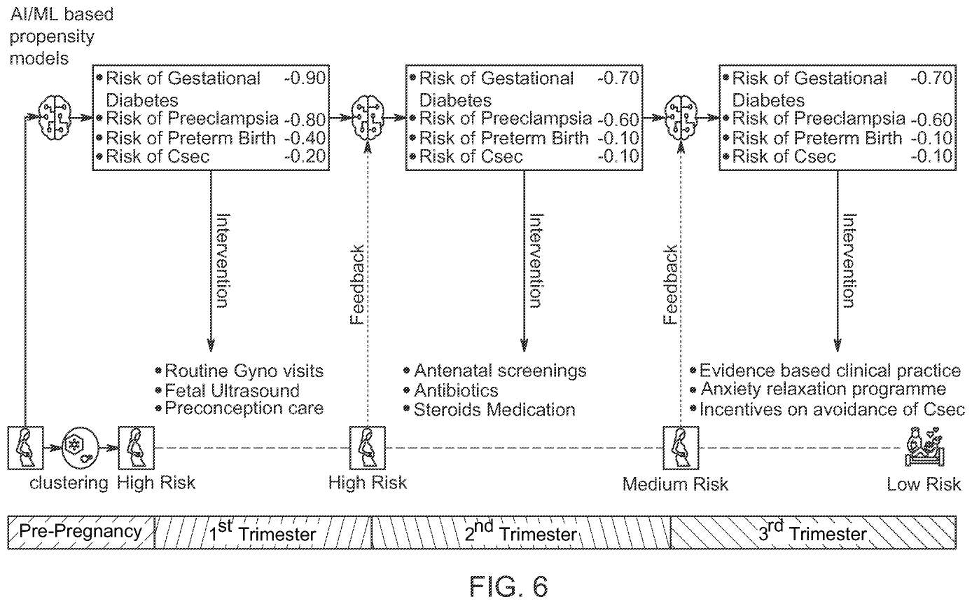

FIG. 6 illustrates an exemplary intervention strategy determination for monitoring the pregnancy of a plurality of subject patients according to an embodiment herein. The system identifies the risk personas at time of attaining pregnancy which are then assigned with corresponding interventions. Further, the system is adapted to change the intervention strategies based on changing risk personas of the pregnant women, generate a weekly report generation and transfer the report to a care provider to focus on higher risk profiles based on changing risk personas, gather feedback mechanism based on changing risk personas and recommend a planned action for each pregnancies, calculate the risk for delivery and support the providers with precautionary measures prior to planning of delivery and delivery type.

In some embodiments, as illustrated in FIG. 6, the pregnancy period includes a pre-pregnancy, 1st trimester, 2nd trimester and 3rd trimester. The care monitoring process works using AI/ML based propensity models which receives data sets 125 in the form of medication, SODH and previous insurance claim history, and determines various risks associated to the subject patients. The method further includes clustering all eligible population of female basis at the time of attaining pregnancy and identifies various risk factors including one or more high-risk factor, one or more medium risk factors, and one or more low risk factors. The high-risk factor is identified based on routine visits to gynecologist, fetal ultrasound and preconception care and the feedback is transferred to the propensity models. In some embodiments, in high risk factor, the risk of Gestational Diabetes is 0.90, Risk of Preeclampsia is 0.80, Risk of Preterm Birth is 0.40 and Risk of C-sec is 0.20. The medium-risk factor is identified based on Antenatal screenings, Antibiotics and Steroids Medication and the feedback is transferred to the propensity models. In some embodiments, in medium risk factor the Risk of Gestational Diabetes is 0.70, Risk of Preeclampsia is 0.60, Risk of Preterm Birth is 0.10, Risk of C-sec is 0.10. The low-risk factor is identified based on Evidence based clinical practice, Anxiety relaxation program and incentives on avoidance of C-sec. In some embodiments, in low risk factor the Risk of Gestational Diabetes is 0.70, Risk of Preeclampsia is 0.60, Risk of Preterm Birth is 0.10 and Risk of C-see is 0.10.

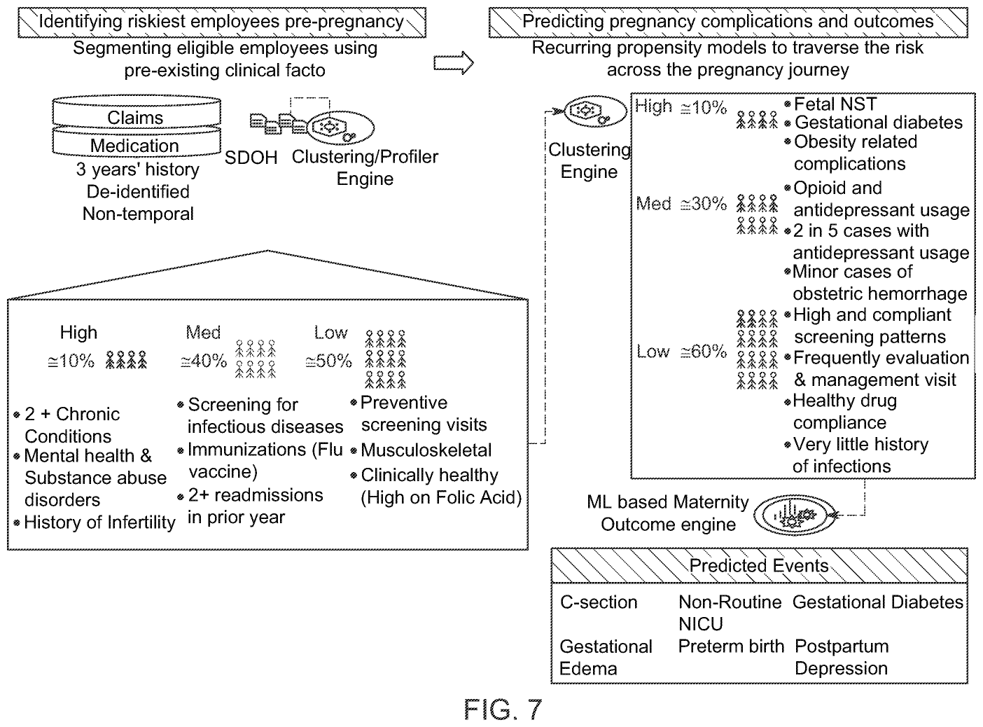

Yet in another embodiment, as illustrated in FIG. 7, the method includes segmenting the eligible employee using pre-existing clinical factors. The pre-existing clinical factors may be captured and used for the maternity risk prediction, even when the user is not planning for maternity or not in the maternity phase. The risk factor is classified as high, medium and low. The high-risk factor includes one or more chronic conditions, a mental health and substance abuse disorders and a history of infertility. The medium factor includes a screening of infectious diseases, an immunizations (Flu vaccine) and a readmissions in prior year. The low factor includes preventive screening visits, a musculoskeletal, and being clinically healthy (High on Folic Acid). As illustrated, the 10% of women are at high risk factor, 40% of women are at medium risk factor and 50% of women are at low risk factor. The predicting method includes recurring propensity models to traverse the risk across the pregnancy journey. The method for predicting pregnancy complication is classified as high, medium and low. The high complication includes a fetal NST, a gestational diabetes and an obesity related complication. The medium complication includes opioid and antidepressant usage, 2 in 5 cases with antidepressant usage and a minor cases of obstetric hemorrhage. The low complication includes high and compliant screening patterns, frequent evaluation and management visits, healthy drug compliance and very little history of infections. In some embodiments, 10% of women are predicted as high complication, 30% of women are predicted as medium complication and 60% of women are predicted as low complication. In some embodiments, the predicted events are C-section, Non-Routine NICU, Gestational Diabetes, Gestational Edema, Preterm birth and Postpartum Depression.

The present disclosure may be a system, a method, and/or a computer program product. The computer program product may include a computer readable storage medium (or media) having computer readable program instructions thereon for causing a processor to carry out aspects of the present disclosure. In some embodiments, the system is used in collaboration with employee management systems, for identifying riskiest employees' pre-pregnancy and predicting pregnancy complication. In such embodiments, the system calculates a risk for all eligible population of female basis at the time of attaining pregnancy considering historical illness and other socio-economic risks and categorization of the pregnant women into risk basis clusters based on the patient personal data, evaluates the risk persona continuously using ongoing hormonal, other behavioral and responsive changes to estimate the risk migration through multiple trimesters of pregnancy, evaluates multiple risk components using advanced analytics algorithms on daily basis to pop out alerts for the corresponding provider, generates a weekly predictive risk reports for patients and forward it to corresponding specialist for manual verification, calculates the multi risk factors during pregnancy and their probability of occurrence and generating at least one of a risk of preterm birth or having a cesarean delivery at the point of birth time. This is to be understood, that the apparatus of the current disclosure is equally applicable for the purpose of determination of maternity risks and/or any other risks in a user who is not currently undergoing any stages of pregnancy without limiting to any age, period, demographics, profile user thereof.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to individualize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments of the present disclosure have been described, the technical scope of the disclosure is not limited to the above described embodiments. It is apparent to people skilled in the art that various alterations and improvements can be added to the above-described embodiments. It is also apparent from the scope of the claims that the embodiments added with such alterations or improvements can be included in the technical scope of the disclosure.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

As made clear from the above, the embodiments of the present disclosure enable a learning apparatus learning a model corresponding to time-series input data to have higher expressive ability and learning ability and to perform the learning operation more simply.

We claim:

1. A system for identifying a maternity severity index of a subject user, the system comprising:
a computing unit, the computing unit comprising an input/output unit adapted to present or formulate at least one query based at least in part on one or more of health states, socio-economic states, or behavioral states of said subject user, said at least one query being configured to elicit at least one response from said subject user; and
a backend server communicably connected to the computing unit via a communication medium, the back-end server comprising:
a data receiving component adapted to receive data-sets from one or more data sources, the received data-sets pertaining to at least one risk associated to maternity within the subject user,
a constantly upgraded risk assessment database comprising at least one of a plurality of sample maternity data parameters, assessments, risk evaluations, suggested recommendations or intervention plans for treating said at least one risk from a plurality of users of different ages, demographic regions, ethnicity, psycho or social-economic, health, and biological variables, and
a maternity risk assessment module adapted to assess the at least one risk associated to said subject user, the maternity risk assessment module being configured to determine at least one of threats or a maternity severity index related to the subject user using the received data-sets, the maternity risk assessment module comprising one or more programming instruction causing the computing unit to:
determine a current risk persona of the subject user on a basis of response of the subject user to the at least one query, the received data-sets, and the sample maternity data parameters, the risk persona being categorized in a form of one or more risk-based clusters,
calculate a series of predicted maternity risk realizations for a predetermined future time span by at least partially correlating the response of the subject user to the query with the sample maternity data parameters,
generate a risk report for the predetermined time period, the report at least determining a risk possibility of at least one of a preterm birth or a cesarean delivery at a point of a birth time, and
generate an intervention strategy adapted to reduce a possibility of maternity risk realizations.

2. The system of claim 1, further comprising a visualization generation component comprising a user interface graphically depicting the at least one risk or the at least one threat onto an interactive dashboard.

3. The system of claim 1, further comprising a data sharing component adapted to share the risk report with external systems via an application programming interface (API).

4. The system of claim 1, wherein the received data-sets comprise maternity related data selected from one or more of insurance claims data, medication data, or behavioral data.

5. The system of claim 1, wherein the risk assessment module comprises a plurality of sub-modules, each catering to a different perspective related to the maternity within the subject user.

6. The system of claim 1, wherein the step of determining the current risk persona comprises a data correlation to draw a relation between user submitted data, the received data-sets from the one or more data sources and a maternity sample database.

7. The system of claim 1, wherein the subject user is in a state of maternity selected from at least one of: pre-maternity, first trimester, a second trimester, a third trimester, a post pregnancy period.

8. The system of claim 1, wherein the subject user is not in a state of maternity.

9. The system of claim 1, wherein the one or more risk-based clusters comprise one or more categories defined in accordance with a severity level thereof.

10. The system of claim 1, wherein the intervention strategy comprises a plurality of: recommendations, lifestyle plans, or therapy plans suitable for the subject user based on the risk predicted by the risk assessment module.

11. The system of claim 6, wherein the programming instructions are further configured to assess an assessment result of said intervention strategy onto the subject, wherein said assessment result is added to the sample maternity database.

12. The system of claim 1, wherein the programming instructions are based on a learning model selected from are selected from one or more of: a natural language processing (NLP) model, a deep learning algorithm model, or a statistical model.

13. The system of claim 11, wherein the programming instructions are improvised or trained with the assessment result from the subject user and/or from the plurality of users.

14. The system of claim 1, wherein the computing unit comprises a mobile, touch-based computing hardware selected from at least one of: a smart phone or a tablet.

15. A method of identifying a maternity severity index of a subject user, the method comprising:
providing a computing unit, the computing unit comprising an input/output unit adapted to present or formulate at least one query based at least in part on one or more of health states, socio-economic states, or behavioral states of said subject user, said at least one query being configured to elicit at least one response from said subject user;
providing a backend server communicably connected to the computing unit via a communication medium, the back-end server comprising:
a data receiving component adapted to receive data-sets from one or more data sources, the received data-sets pertaining to at least one risk associated to maternity within the subject user,
a constantly upgraded risk assessment database comprising at least one of a plurality of sample maternity data parameters, assessments, risk evaluations, suggested recommendations or intervention plans for treating said at least one risk from a plurality of users of different ages, demographic regions, ethnicity, psycho or social-economic, health, and biological variables, and
a maternity risk assessment module adapted to assess the at least one risk associated to said subject user, the maternity risk assessment module being configured to determine at least one of threats or a maternity severity index related to the subject user using the received data-sets; and
executing one or more programming instructions within the maternity risk assessment, the one or more programming instructions for:
determining a current risk persona of the subject user on a basis of response of the subject user to the at least one query, the received data-sets, and the sample maternity data parameters, the risk persona being categorized in a form of one or more risk-based clusters,
calculating a series of predicted maternity risk realizations for a predetermined future time span by at least partially correlating the response of the subject user to the query with the sample maternity data parameters,
generating a risk report for the predetermined time period, the report at least determining a risk possibility of at least one of a preterm birth or a cesarean delivery at a point of a birth time, and
generating an intervention strategy adapted to reduce a possibility of maternity risk realizations.

16. The method of claim 15, wherein the step of determining the current risk persona comprises using a data correlation to draw a relation between user submitted data, the received data-sets from the one or more data sources and a maternity sample database.

17. The method of claim 15, wherein the programming instructions further comprise instructions for assessing an assessment result of said intervention strategy onto the subject, wherein said assessment result is added to the sample maternity database.

18. The method of claim 17, wherein the programming instructions are improvised or trained with the assessment result from the subject user and/or from the plurality of users.

19. The method of claim 15, wherein the programming instructions are based on a learning model selected from are selected from one or more of: a natural language processing (NLP) model, a deep learning algorithm model, or a statistical model.
